(19) United States
(12) Patent Application Publication
(10) Pub. No.: US 2022/0022788 A1
(43) Pub. Date: Jan. 27, 2022

(54) SEASONAL AFFECTIVE DISORDER DETERMINATION

(71) Applicant: Medtronic, Inc., Minneapolis, MN (US)

(72) Inventors: Bruce D. Gunderson, Plymouth, MN (US); Trent M. Fischer, St. Paul, MN (US)

(21) Appl. No.: 16/940,032

(22) Filed: Jul. 27, 2020

(51) Int. Cl. A61B 5/16; G16H 40/67; G16H 50/30; G16H 50/70; G16H 50/50; A61B 5/00; A61B 5/0205; A61B 5/11

(52) U.S. Cl. CPC A61B 5/165; G16H 40/67; G16H 50/30; G16H 50/70; G16H 50/50; A61B 5/746; A61B 5/024; A61B 5/0205; A61B 5/4815; A61B 5/1118; A61B 5/4809; A61B 5/4836; A61B 5/4848; A61B 5/686

(57) ABSTRACT

Techniques are disclosed for detecting when a patient is experiencing seasonal affective disorder (SAD). An example computer-implemented method includes determining a status of one or more health metrics for a patient based on sensor data collected from at least one biometric device. The method also includes determining potential sun exposure for a patient based on weather metrics corresponding in time with the health metrics. Additionally, the method includes, when a decline in the status of the health metrics for the patient correlates with declining potential sun exposure, providing an alert indicative that patient is experiencing SAD.

### TECHNICAL FIELD

The invention relates to medical device systems and, more particularly, medical device systems for monitoring a condition of a patient.

### BACKGROUND

Some types of implantable medical devices (IMDs) may be used to monitor one or more physiological parameters of a patient, such as physiological parameters associated with cardiac or pulmonary function. Such IMDs may include, or may be part of a system that includes, sensors that detect signals associated with such physiological parameters; e.g., tissue impedance or oxygen levels. Values determined based on such signals may be used to assist in detecting changes in medical conditions, in evaluating the efficacy of a therapy, or in generally evaluating patient health.

Implantable devices that monitor physiological parameters related to a medical condition of a patient may evaluate values associated with the physiological parameters, such as to determine whether the values exceed a threshold or have changed. Values that exceed a threshold or that have changed may indicate that a therapy being administered to the patient is not effectively managing the patient's medical condition.

### SUMMARY

In general, the techniques of this disclosure include detecting when a patient is experiencing seasonal affective disorder (SAD) based on health metrics measured by a biometric monitoring device and weather metrics gathered from a networked weather repository. A monitor, via the device, measures and/or determines health metrics, such as daytime activity, sleep duration, sleep quality, night oxygen concentration, and night minimum heart rate. The monitor retrieves and/or otherwise receives weather metrics (e.g., taken from a weather service) based on the location of the patient (e.g., using phone-based location data, Internet Protocol (IP) address geolocation, etc.), such as atmospheric pressure, daylight hours, temperature, precipitation, and/or percent sun, etc. When the monitor detects a sufficient correlation (e.g., a threshold correlation coefficient for a predetermined amount of time, etc.) between a decline in one or more of the health metrics and adverse weather metrics indicative of lower sun exposure, the monitor provides an alert to the user and/or a physician of the user to, for example, initiate treatment for SAD.

An example computer-implemented method includes determining a status of one or more health metrics for a patient based on sensor data collected from at least one biometric device. The method also includes determining potential sun exposure for a patient based on weather metrics corresponding in time with the health metrics. Additionally, the method includes, when a decline in the status of the health metrics for the patient correlates with reduced potential sun exposure, providing an alert indicative that patient is experiencing SAD.

An example system includes memory to store health metrics and weather metrics and processing circuitry. The processing circuitry determines a status of one or more of the health metrics for a patient based on sensor data collected from at least one biometric device. The processing circuitry also determines potential sun exposure for a patient based on the weather metrics corresponding in time with the health metrics. Additionally, when a decline in the status of the health metrics for the patient correlates with reduced potential sun exposure, the processing circuitry provides an alert indicative that patient is experiencing SAD.

An example computer readable medium comprising instructions that, when executed cause processing circuitry to determine a status of one or more of the health metrics for a patient based on sensor data collected from at least one biometric device. The instructions also cause the processing circuitry to determine potential sun exposure for a patient based on the weather metrics corresponding in time with the health metrics. Additionally, the instructions cause the processing circuitry to, when a decline in the status of the health metrics for the patient correlates with reduced potential sun exposure, provide an alert indicative that patient is experiencing SAD.

The details of one or more examples are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating a system configured to monitor for seasonal affective disorder (SAD) in a patient, in accordance with some techniques of this disclosure.

FIG. 2 is a block diagram illustrating an example computing device configured to monitor for SAD, in accordance with some techniques of this disclosure.

FIG. 3 is a flow diagram illustrating an example method of determining whether to provide an indication that a patient is experiencing SAD, in accordance with techniques of this disclosure.

FIG. 4 is a flow diagram illustrating an example method to evaluate effectiveness of therapy for SAD, in accordance with techniques of this disclosure.

### DETAILED DESCRIPTION

Seasonal affective disorder (SAD) is a type of depressive or bipolar disorder with recurrent affective episodes. Frequently, a patient will experience SAD during the fall and winter seasons. Between 1% and 10% of the population experiences SAD. Patients with SAD may experience difficulty sleeping, depressive episodes, difficulty concentrating, changes in motivation and energy, and/or changes in appetite or weight, etc. Additionally, SAD has a significant impact on patients with coronary heart disease or risk of coronary heart disease. For example, SAD may increase the risk of an adverse cardiac event such as a heart attack or blood clots. For patient at risk of coronary heart disease, SAD may also increase the risk of a heart attack and development of coronary artery disease. There are therapies for SAD, such as light therapy, psychotherapy, exercise, regular sleep times, and medications, etc. For example, bright light therapy via a light box may be effective to ease symptoms of SAD. However, patients may not realize that they are experiencing SAD or because of the symptoms, may not be motivated to discuss their symptoms with a clinician.

As described below, a monitor collects biometric data (sometimes referred to as “health metrics”). The monitor is communicatively coupled to one or more biometric data collection devices. The monitor also periodically (e.g., daily) collects weather data corresponding in time with the biometric data. The monitor tracks the trend of the health metrics to determine when the health metrics are declining. The monitor calculates, based on the weather metrics, potential sunlight exposure. When the monitor detects a correlation between declining health metrics and low sunlight exposure, the monitor provides an alert to the patient and/or the patient's clinician of a potential occurrence of SAD. In some examples, the monitor may provide the alert after the correlation has been within a threshold value for a threshold period of time (e.g., fifteen days, thirty days, etc.). Subsequently, upon undertaking therapy, the monitor may, based on the health metrics and the weather metrics determine effectiveness of the therapy. When the therapy is not effective, the monitor may provide an alert

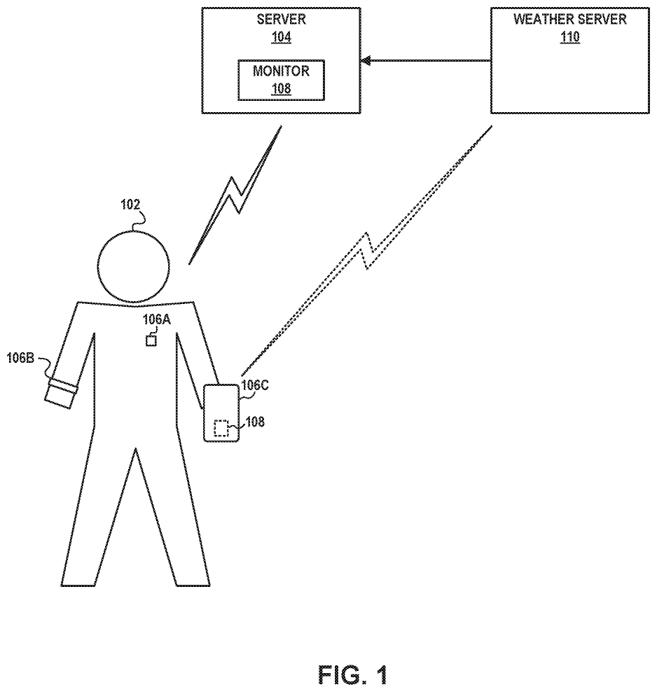

FIG. 1 is a schematic diagram illustrating a system 100 configured to monitor for seasonal affective disorder (SAD) in a patient 102. System 100 includes a server 104 configured to determine whether patient 102 likely has SAD, based on data collected by one more biometric devices 106A-106C (collectively “biometric devices 106”). Biometric devices 106A-106C may include a wide variety of different types of devices with sensors configured to collect various data indicative of one or more physical parameters or behaviors of patient 102. For example, biometric devices 106A-106C may include an implantable sensor device 106A within the body of patient 102, a wearable sensor device 106B (e.g., such as a fitness tracker, a smart watch, etc.) worn by patient 102, or any other device 106C that measures physical parameters or behaviors of patient, such as a smartphone. Wearable sensor device 106B may be a wrist-wearable activity monitor, including one or more accelerometers (inertial measurement unit, or IMU), pedometers, optical sensors (photoplethysmography (PPG) sensors), and/or other sensors. In some examples, wearable sensor device 106B may include a patch configured to be attached to patient 102 at another location such as on the chest of patient 102.

An implantable sensor device 106A may take the form of an implantable medical device (IMD), such as a cardiac monitor having electrodes configured to collect data, such as a subcutaneous electrocardiogram (ECG) signal and/or a cardiac electrogram (EGM) signal indicative of electrical activity of a heart of patient 102, including data regarding heart rate, heart rate variability, and arrhythmic episodes. IMD 106A may also be configured with one or more sensors to collect other physiological data, such as one or more accelerometers configured to detect movement, steps, and posture/orientation, one or more temperature sensors, electrodes to sense respiration or mechanical activity of the heart, or one or more optical sensors (e.g., PPG sensors) to sense oxygen saturation or mechanical activity of the heart.

One example of a cardiac monitor is the Reveal LINQ™ Insertable Cardiac Monitoring System, available from Medtronic plc. The Reveal LINQ™ Insertable Cardiac Monitoring System is an example of a cardiac monitor that includes electrode configured to sense a subcutaneous ECG, as well as other sensors. Other examples of implantable sensor device 16 include devices configured as pacemakers, cardioverters, and/or defibrillators, which may include one or more electrodes positioned on, within, or near the heart, e.g., via one or more leads, to sense a cardiac EGM. Such devices may include additional sensors as described herein.

System 100 includes server 104 configured to receive sensor data from any or all of biometric devices 106A-106C. Server 104 includes memory and processing circuitry configured to receive sensor data and process the data according to the techniques of this disclosure. Server 104 may be a remote server, such as managed by a medical practice or practitioner, or a manufacturer of one of biometric devices 106A-106C, such that a physician for patient 102 may access and view the data so as to inform treatment of patient 102 as needed. For example, server 104 may operate on a cloud server and may be incorporated into a medical online patient portal. While described as operating on server 104, the techniques of this disclosure may be performed on other computing devices, such as a personal computing device, smartphone, tablet, or laptop. In some examples, some or all of the functionality described herein as being performed by server 104 (e.g., by processing circuitry of the computing device) may be performed by one or more of biometric devices 106A-106C (e.g., by processing circuitry of the one or more biometric devices). For example, one or more of biometric devices 106A-106C may independently or cooperatively identify when patient 102 is sleeping, identify duration of a sleep state, identify interruptions of the sleep state, determine a quality metric for the sleep states, and report numbers and/or rates of these metrics to server 104.

In the illustrated example, server 104 includes a monitor 108. Monitor 108 receives sensor data from biometric devices 106A-106C, and process the data in order to qualify and/or quantify health metrics. These health metrics include daytime activity, sleep duration, sleep quality, nighttime oxygen, and/or nighttime heart rate, etc. Monitor 108 determines when the health metrics are indicative in a decline in health of the patient. For example, monitor 108 may determine the health metrics are indicative in a decline in health based on a decrease in daytime activity, decreased in sleep quality, increase in minimum night heart rate, and/or an increased night oxygen drops. In some examples, decline in the health metrics may be determined based on whether or not the health metric satisfies a threshold (e.g., an absolute threshold, a threshold personalized for patient 102, etc. Daytime activity may be measured in minutes of activity that satisfies a threshold level of activity, based on, for example, heart rate of patient 102 and/or movement of patient 102. Sleep quality may be measured by amount movement of patient 102 during resting hours or after patient 102 has otherwise been determined to be asleep or attempting to sleep. For example, monitor 108 may determine that the night oxygen metric has declined when oxygen percentage drops 4% while patient 102 is sleeping or when the oxygen percentage is below 90%). In some examples, a decline in the health metrics may be determined by a linear change over time. For example, a linear decrease in sleep quality over time (e.g., as evidenced by increased nighttime movement, etc.) may be indicative of a decline in the sleep quality health metric even if the metric does not drop below an absolute threshold. As another example, a linear decrease in daytime activity minutes may be indicative of a decline in daytime activity. For example, a linear increase in nighttime heart rate may indicate a decline in the health metrics. As used herein, determining a decline in health metrics does not necessarily mean that metrics are continually getting worse. Rather, decline in health metrics means that a predetermined number of the health metrics either (i) satisfy, e.g., are below or above, a corresponding threshold that is indicative of a decline in the health of patient 102, and/or (ii) are experiencing a linear change in a manner that is indicative of a decline in the health of patient 102. In some examples, monitor 108 may determine the health metrics are declining when all of the measured health metrics (e.g., daytime activity, sleep duration, sleep quality, nighttime oxygen, and/or nighttime minimum heart rate, etc.) satisfy corresponding thresholds. Alternatively in some examples, monitor 108 may determine the health metrics are declining when a threshold number of the health metrics satisfy corresponding thresholds.

In some examples, the threshold values form one or more of the health metrics may be individualized to patient 102 to, for example, account for the medical history of patient 102. Health metric threshold values may be set by a clinician. Health metric threshold values may be based, for example, on baseline values for the patient (e.g., baseline resting heartrate, average sleep duration, etc.) and/or demographic factors (e.g., age, gender, etc.). In some examples, health metrics may be adjustable over time based on previous values of the metric, e.g., the threshold may be based on a mean or median of N values, either most recent or from some other time prior to the current time. In this manner, the threshold could track slight changes over time but be satisfied by a recent significant decline in condition

Monitor retrieves weather metrics corresponding in time to the health metrics from one or more weather servers 110. Weather servers 110 are maintained by any suitable entities with an interest in weather, such as a government agency (e.g., the National Weather Service, the National Oceanic and Atmospheric Administration, etc.) and/or a commercial weather forecast provider (e.g., AccuWeather®, Weather Underground®, etc.), In some examples, monitor 108 accesses data from the weather servers 110 via application programming interfaces (APIs) provided by the managing entity. Monitor 108 may use global positioning system (GPS) coordinates obtained from wearable sensor device 106B and/or smartphone 106C to determine the weather to associated with patient 102. Alternatively or addition, in some examples, monitor may use a geographic identifier (e.g., a zip code, a municipality, a census tract, etc.) associated with patient 102 to determine the weather associated with patient 102. Weather metrics collected by monitor 108 includes atmospheric pressure, daylight hours, minimum temperature, maximum temperature, median temperature, heat index, precipitation, cloud cover, and/or percent sun, etc. Using the weather metrics, monitor 108 determines a potential sun exposure for patient 108. In some examples, potential sun exposure is daylight hours sunrise to sunset, discounting hours (e.g., weighing, etc.) that are associated with (i) adverse sun exposure events, such as precipitation, cloud cover, (ii) temperature extremes (e.g., temperatures below freezing, temperatures above 100 degrees Fahrenheit, etc.), and/or (iii) angle of incident of the sun to the surface (e.g., near sunrise and sunset, etc.), etc. In some examples, weights assigned to each hour may depend on demographics and/or a mobility score of patient 102 and/or on temperature deviations from a temperate climate (e.g., patient 102 may be less likely to be outside when temperatures are outside of comfortable range, etc.).

Periodically (e.g., daily, weekly, etc.), monitor 108 performs a correlation between declining health metrics and potential decreasing sun exposure. The health metrics and potential sun exposure are time series of daily values. These time series of numbers are correlated using cross-correlation. The cross correlation determines whether the metrics are correlated in order to trigger an alert. In some example, monitor 108 determines that the declining health metrics and potential sun exposure are correlated when the correlation coefficient (sometime referred to as “R”) is greater than 0.8. In some examples, monitor 108 may determine that patient 102 is experiencing SAD when the correlation is maintained for a threshold period of time (e.g., 15 days, 30 days, etc.). Upon determining that patient 102 is likely experiencing SAD, monitor 108 provides an alert. In some examples, monitor 108 provides the alert to wearable sensor device 106B and/or smartphone 106C. Alternatively or additionally, in some examples, monitor 108 provides the alert to a physician or clinician associated with treatment of patient (e.g., cardiac treatment, mental health treatment, etc.).

The physician or clinician may provide treatment in response to the alert. For examples, the physician or clinician may direct patient to engage in light therapy, take vitamin D supplements, make dietary adjustments, increase exercise, and/or proscribe medications. Subsequently, monitor 108 may continue to monitor health metrics of patient 108 and the weather metrics to determine whether the therapy is effective. Monitor 108 determines whether there is an inverse correlation with the health metrics and the weather metrics (e.g., health metrics improve while sun exposure continues to decrease, health metrics improve while potential sun exposure remains under the threshold level, etc.) or the positive correlation is not present any longer (e.g. R<0.4). When there is an inverse correlation, monitor 108 may send a message to patient 102 and/or the physician that the therapy is effective. When the health metrics do not improve after a threshold period of time (e.g., 15 days, 30 days, etc.), monitor 108 may send a message to patient 102 and/or the physician that the therapy is not effective.

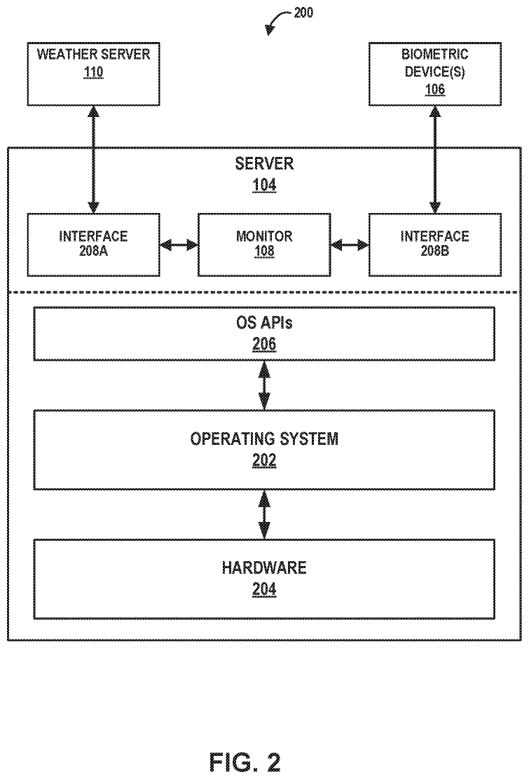

FIG. 2 is a block diagram of example electronic components 200 of the server 104. In the illustrated example, the electronic components 200 includes a device operating system 202 for controlling device hardware resources 204 (e.g., processor(s), memory, network interfaces, etc.) and managing various system level operations, operating system APIs 206 used as interfaces between operating system 202, and a network interfaces 208A and 208B to communicate with the weather service 110 and biometric devices 106 respectively.

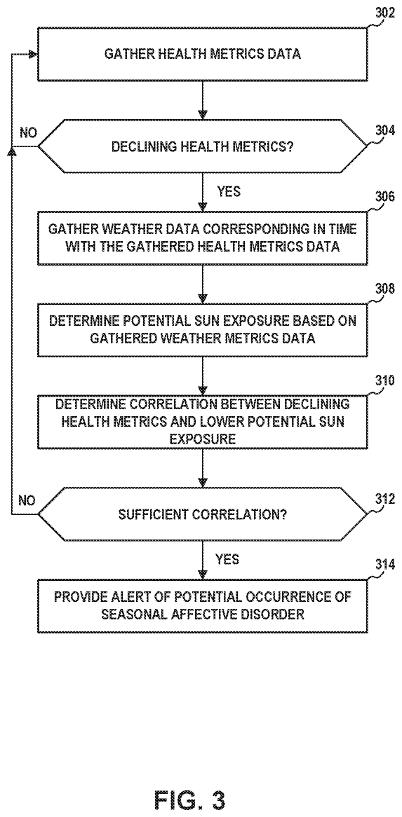

FIG. 3 is a flow diagram illustrating an example method of determining whether to provide an indication that patient 102 is experiencing SAD, in accordance with techniques of this disclosure. Initially, monitor 108 gathers health metrics data of patient 102 (302). Monitor 108 may gather the health metrics data periodically (e.g., hourly, daily, weekly, etc.). Monitor 108 determines where there is a decline in the health metrics (304). Monitor 108 determines there is a decline in the health metrics when a threshold number of health metrics satisfy a threshold value. For example, monitor 108 may determines that there is a decline in the health metrics when nighttime blood oxygen levels fall below 90 percent, sleep duration falls by 25 percent from a baseline established for patient 102, and minutes of activity declines at least 33% from a baseline established based on the age of patient 102, etc. or there is a significant linear decline in one or more of the parameters over a certain period of time. When monitor 108 determines that there is not a decline in the health metrics (“NO” at 304), monitor 108 continues to periodically collect health metric data (302). When monitor 108 determines that there is a decline in the health metrics (“YES” at 304), monitor 108 gather weather data corresponding in time with the gathered health metrics (306). For example, when monitor 108 gathers health metrics daily, monitor gathers weather metrics for the corresponding day. Monitor 108 determines potential sun exposure based on the gathered weather metrics data (308). Monitor 108 determines correlation between declining daily health metrics and daily sun exposure (310). Monitor 108 determines whether there is sufficient cross correlation between declining daily health and daily sun exposure (312). For examples, there may be sufficient correlation when health metrics have declined on days with potential sun exposure is below the threshold. In some examples, there may be sufficient correlation when health metrics follow potential sun exposure (e.g., days with low potential sun exposure also have declining health metrics and days have at least a baseline level of potential sun exposure also have non-declining health metrics, etc.). In some examples, sufficient correlation includes a threshold number of days that health metrics and weather metrics are correlated. When there is sufficient correlation (“YES” at 312), monitor 108 provides an alert that patient 102 is potentially experiencing SAD (314). Otherwise, when there is not sufficient correlation (“NO” at 312), monitor 108 continues to periodically collect health metric data (302).

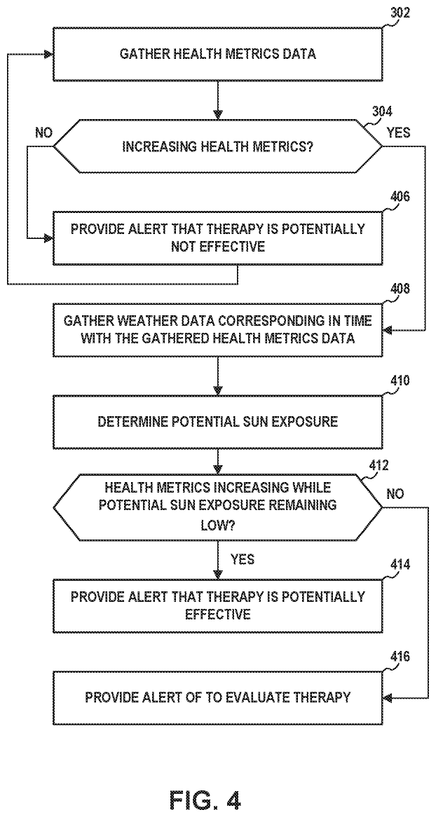

FIG. 4 is a flow diagram illustrating an example method to evaluate effectiveness of therapy for SAD, in accordance with techniques of this disclosure. After receiving an alert (e.g. the alert generated at 314 of FIG. 3, etc.) that patient 102 may be experiencing SAD, a clinician may set a therapy plan with patient 102. For example, the clinician may set a therapy plan that includes light therapy, vitamin D supplements, a dietary change, an activity level change, counseling, and/or medicine. Initially, monitor 108 gathers health metrics data of patient 102 (402). Monitor 108 may gather the health metrics data periodically (e.g., hourly, daily, weekly, etc.). Monitor 108 determines where there is an increase in the health metrics (404). Monitor 108 may gather data from a threshold period of time before making a determination whether there is an increase in the health metrics. For example, the monitor may gather health metrics for twenty days post treatment plant before making a determination. Health metrics may be increasing when at least one health metric no longer satisfies the threshold for determining the health metric is declining. When there is no increase in health metrics (“NO” at 404), monitor 108 provides and alert that therapy is potentially not effective (406).

When there is an increase in health metrics (“NO” at 404), monitor 108 gather weather day corresponding in time with the gather health metrics data (408). Monitor 108 determine potential sun exposure from the weather metrics data (410). Monitor 108 determines whether health metrics are increasing while potential sun exposure remain low or is decreasing (412). When health metrics are increasing while potential sun exposure remain low or is decreasing (“YES” at 412), monitor 108 provides an alert that the therapy plan is potentially effective (414). Otherwise, when health metrics are increasing while potential sun exposure remain is increasing (“NO” at 412), monitor 108 provides an alert to evaluate therapy (416).

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable medium may cause a programmable processor, or other processor, to perform the method, e.g., when the instructions are executed. Computer-readable media may include non-transitory computer-readable storage media and transient communication media. Computer readable storage media, which is tangible and non-transitory, may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer-readable storage media. It should be understood that the term “computer-readable storage media” refers to physical storage media, and not signals, carrier waves, or other transient media.

Various examples of the invention have been described. These and other examples are within the scope of the following claims.

What is claimed is:

1. A computer-implemented method comprising:
determining a status of one or more health metrics for a patient based on sensor data collected from at least one biometric device;
determining potential sun exposure for a patient based on weather metrics corresponding in time with the health metrics; and
when a decline in the status of the health metrics for the patient correlates with declining potential sun exposure, providing an alert indicative that patient is experiencing seasonal affective disorder (SAD).

2. The method of claim 1, wherein the at least one biometric device includes an implantable medical device.

3. The method of claim 1, wherein the one or more health metrics for the patient include one or more of daytime activity, sleep duration, sleep quality, nighttime oxygen, or nighttime minimum heart rate.

4. The method of claim 1, wherein the weather metrics includes at least one of atmospheric pressure, daylight hours, minimum temperature, maximum temperature, median temperature, precipitation, and cloud cover.

5. The method of claim 1, comprising determining that the status of the health metrics is in decline when all of the health metrics satisfy a corresponding threshold.

6. The method of claim 1, comprising determining that the status of the health metrics is in decline when a majority of the health metrics satisfy a corresponding threshold.

7. The method of claim 1, wherein providing an alert indicative that patient is experiencing SAD includes determining that the decline in the status of the health metrics for the patient correlates with reduced potential sun exposure for a threshold period of time.

8. The method of claim 1, comprising, subsequent a therapy being provided in response to the alert:
determining the status of health metrics for a patient based on sensor data collected from at least one biometric device;
determining potential sun exposure for a patient based on weather metrics corresponding in time with the health metrics; and
determining whether the status of health metrics is indicative of improvement while the patient is experiencing the reduced potential sun exposure.

9. The method of claim 8, comprising, when the status of health metrics is not indicative of improvement, providing a second alert indicative that the therapy is not effective.

10. A system comprising:
memory to store health metrics and weather metrics; and
processing circuitry configured to:
determine a status of one or more of the health metrics for a patient based on sensor data collected from at least one biometric device;
determine potential sun exposure for a patient based on the weather metrics corresponding in time with the health metrics; and
when a decline in the status of the health metrics for the patient correlates with declining potential sun exposure, provide an alert indicative that patient is experiencing seasonal affective disorder (SAD).

11. The system of claim 10, wherein the at least one biometric device includes an implantable medical device.

12. The system of claim 10, wherein the health metrics for the patient include one or more of daytime activity, sleep duration, sleep quality, nighttime oxygen, or nighttime minimum heart rate.

13. The system of claim 10, wherein the weather metrics includes at least one of atmospheric pressure, daylight hours, minimum temperature, maximum temperature, median temperature, precipitation, and cloud cover.

14. The system of claim 10, wherein the processing circuitry is configured to determine that the status of the health metrics is in decline when all of the health metrics satisfy a corresponding threshold.

15. The system of claim 10, wherein the processing circuitry is configured to determine that the status of the health metrics is in decline when a majority of the health metrics satisfy a corresponding threshold.

16. The system of claim 10, wherein to provide an alert indicative that patient is experiencing SAD, the processing circuitry is configured to determine that the decline in the status of the health metrics for the patient correlates with reduced potential sun exposure for a threshold period of time.

17. The system of claim 10, wherein the processing circuitry is configured to, subsequent a therapy being provided in response to the alert:
determine the status of health metrics for a patient based on sensor data collected from at least one biometric device;
determine potential sun exposure for a patient based on weather metrics corresponding in time with the health metrics; and
determine whether the status of health metrics is indicative of improvement while the patient is experiencing the reduced potential sun exposure.

18. The system of claim 17, wherein the processing circuitry is configured to, when the status of health metrics is not indicative of improvement, provide a second alert indicative that the therapy is not effective.

19. A computer readable medium comprising instructions that, when executed cause processing circuitry to:
determine a status of one or more of the health metrics for a patient based on sensor data collected from at least one biometric device;
determine potential sun exposure for a patient based on the weather metrics corresponding in time with the health metrics; and
when a decline in the status of the health metrics for the patient correlates with reduced potential sun exposure, provide an alert indicative that patient is experiencing seasonal affective disorder (SAD).

20. The computer readable medium of claim 19, wherein the instructions further cause the processing circuitry to, subsequent a therapy being provided in response to the alert:
determine the status of health metrics for a patient based on sensor data collected from at least one biometric device;
determine potential sun exposure for a patient based on weather metrics corresponding in time with the health metrics; and
when the status of health metrics is indicative of improvement while the patient is experiencing the reduced potential sun exposure, provide a second alert indicative that the therapy is not effective.
